(19) United States
(12) Patent Application Publication
(10) Pub. No.: US 2023/0210574 A1
(43) Pub. Date: Jul. 6, 2023

(54) Method and System for Minimally Invasive Removal of Mesenteric Fat

(71) Applicant: B2M Medical, Inc., Irvine, CA (US)

(72) Inventors: Alexei Babkin, Dana Point, CA (US); Rafi Mazor, San Diego, CA (US)

(73) Assignee: B2M Medical, Inc., Irvine, CA (US)

(21) Appl. No.: 17/568,207

(22) Filed: Jan. 4, 2022

(51) Int. Cl. A61B 18/02; A61B 90/00

(52) U.S. Cl. CPC A61B 18/02; A61B 90/06; A61B 2018/0225; A61B 2018/00464

(57) ABSTRACT

Methods and devices for reducing visceral fat within the mesenteric structure of the body by cooling visceral fat within the mesentery while leaving arteries, veins, nerves and lymph nodes within the mesentery, and the mesentery membrane, undamaged, and thereafter allowing natural processes of the body to eliminate the cooled visceral fat from the body. The system comprises a pair of flat-faced cooling probes configured for insertion into the abdomen and placement on opposite sides of a section of mesentery for application of cooling power to the mesentery, at temperatures in a range which kills visceral fat cells but does not harm other tissue.

### FIELD OF THE INVENTIONS

The inventions described below relate to the field of visceral fat reduction.

### BACKGROUND OF THE INVENTIONS

Visceral fat is found inside the abdominal cavity and wraps around internal organs, as opposed to subcutaneous fat which is stored just below the skin. Visceral fat, and in particular mesenteric fat, may be found in the abdomen, under the abdominal muscles. Visceral fat is associated with high blood pressure, increased risk of heart disease, insulin resistance and diabetes, stroke, some cancers, and continued presence in the body may contribute to these conditions. Though diet and exercise can help eliminate visceral fat, diet and exercise are not well-tolerated by the typical overweight patient. Visceral fat is more susceptible to destruction by cooling to cold temperatures which do not harm surrounding or nearby tissue such as blood vessels, nerves and lymph nodes. Thus, visceral fat can be killed with cooling to temperatures in the range of +10° C. to −60° C., and preferably in the range of +10° C. to −20° C., or the range of −20° C. to −40° C. If cooling is limited to this range, surrounding or nearby tissue will not be affected. Cryogenically deadened visceral fat will be removed by the body over the course of a few weeks. Methods and systems for cooling visceral fat while leaving surrounding tissue unharmed are disclosed in our prior International Patent Publication WO 2020/061202 (Mar. 26, 2020).

The mesentery, or mesentarium, is an organ that attaches the intestines to the posterior abdominal wall in humans and is formed by the double fold of peritoneum. It helps in storing fat and allowing blood vessels, lymphatics, and nerves to supply the intestines, among other functions.

The mesentery includes sections such as ascending, transverse, descending, and sigmoid mesocolons, the mesoappendix, and the mesorectum. The mesentery includes sheet-like sections that connect the intestines in the abdomen to the abdominal wall. The mesentery comprises two walls of peritoneum (the mesenteric membrane), with various arteries, veins and nerves disposed between the two walls and running through the mesentery to supply various organs. Also, the mesentery stores visceral fat, which, when excessive, leads to the ailments mentioned above.

The visceral/mesenteric fat in the sheet-like layered structure is not readily treated with cooling probes or methods of the prior art.

### SUMMARY

The methods and devices described below provide for reducing visceral fat within the mesenteric structure of the body by cooling visceral fat within the mesentery while leaving arteries, veins, nerves and lymph nodes within the mesentery, and the mesentery membrane, undamaged, and thereafter allowing natural processes of the body to eliminate the cooled visceral fat from the body. The system comprises a pair of flat-faced cooling probes (in the easiest implementation) configured for insertion into the abdomen and placement on opposite sides of a section of mesentery for application of cooling power to the mesentery, at temperatures in a range which kills visceral fat cells but does not harm other tissue (in our prior International Patent Publication WO 2020/061202, we referred to this as a non-ablative cold temperature). The flat-faced cooling probes have tissue-contacting surfaces supplied with cooling power, such as flow of a cooling fluid proximate the surfaces, and the cooling surface of each probe may be provided with positioning transmitters/sensors, operable to transmit and/or receive signal from corresponding transmitters/sensors on the other probe, to aid in determining the degree of alignment of the probes on opposite surfaces of the mesentery. The cooling power applied, and the length of time it is applied, may be determined based on the thickness of the mesentery as determined by positioning sensors embedded in the treatment devices and/or and conventional medical imaging such as optical (endoscopic or direct vision), fluoroscopy or ultrasonic imaging, or may be controlled in response to temperature measurements of the mesenteric structure while applying cooling power.

### BRIEF DESCRIPTION OF THE DRAWINGS

FIG. 1 illustrates the anatomy of a patient, including a portion of the mesentery extending from the duodenojejunal flexure to the ileocecal junction which connects portions of the small intestines to the posterior abdominal wall.

FIGS. 2A and 2B illustrates a method of removing or reducing fat within the mesentery by applying cooling power from both sides of a mesentery sheet (a portion of the mesentery).

FIGS. 3 and 4 illustrates a cooling probe for use in the treatment method.

FIG. 5 is a cross section of the cooling probe.

FIG. 6 illustrates the distal surface of the cooling head of the cooling probe.

The results of the operation are illustrated in FIGS. 7A, 7B and 7C.

### DETAILED DESCRIPTION OF THE INVENTIONS

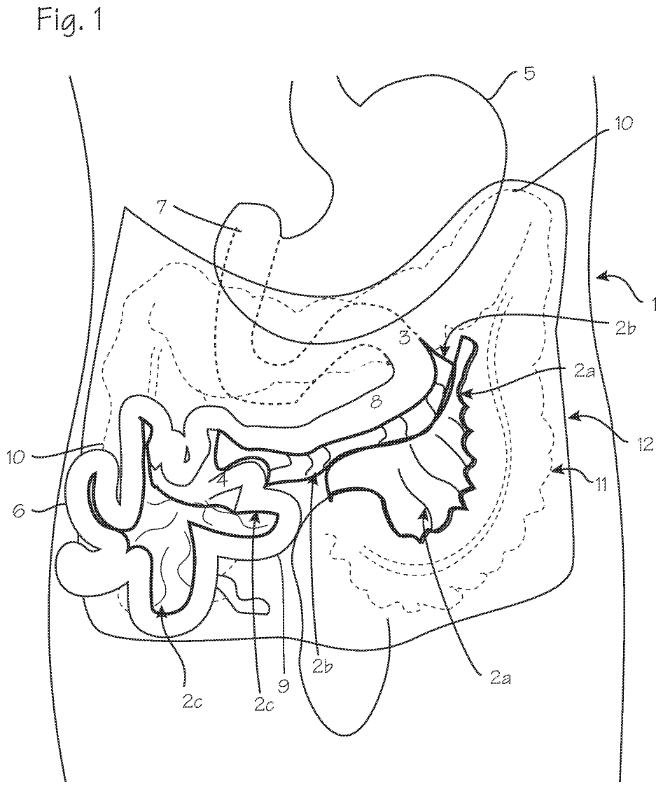

FIG. 1 illustrates the anatomy of a patient 1, including portions of the mesentery 2a, 2b and 2c extending from the duodenojejunal flexure 3 to the ileocecal junction 4 which connects portions of the small intestines to the posterior abdominal wall. This illustration shows the patient's stomach 5, the small intestines 6 which are made up of the duodenum 7, jejunum 8 and the ileum 9, and the large intestines 10 and the colon 11. These are all enclosed within the peritoneum 12, which folds in a complex manner to form the mesentery 2. Several sections 2a, 2b, and 2c of the mesentery that extend from the large intestine (the colon 11), the jejunum 8, and the ileum 9, respectively, are shown. Each sheet extends from the intestine toward the posterior wall of the abdomen. These portions of the mesentery may be fan-shaped, with several folds, and each portion of the small intestines may be attached to a separate sheet-like portion of the mesentery. Other sections of the mesentery connect to the ascending, transverse and descending colon sections of the large intestine, the colon and the appendix.

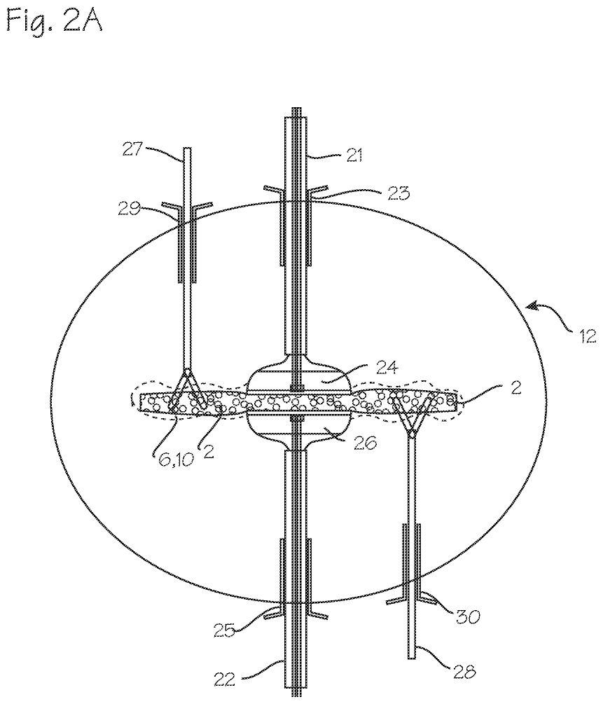

FIGS. 2A and 2B illustrate a method of cooling fat within the mesentery by applying cooling power from both sides of a mesentery sheet (a portion of the mesentery). The portion of the mesentery 2 is shown attached to the large intestines 10 or small intestines 6, and two cooling probes 21 and 22 are shown engaged with the mesentery. One cooling probes 21 has been inserted into the body from a first portal 23 and its cooling head 24 has been placed against one surface of the mesentery, and a second cooling probe 22 has been inserted into the body from a second portal 25 (or the same portal) and its cooling head 26 has been placed against a second, opposing surface of the mesentery. Graspers 27 and 28 may be used to manipulate the intestines and/or mesentery sheet as necessary, and are shown inserted into the abdomen through portals 29 and 30. As shown in FIG. 2B, a section of the intestine (10, 6) has been grasped with graspers 27, 28 and lifted and/or pulled anteriorly so that the attached section of the mesentery 2 hangs down from the intestine (extends posteriorly, depending on the configuration of the mesentery), exposing mesentery surfaces to the cooling heads of the cooling probes. The cooling probes 21 and 22 have been inserted into the abdomen, and the cooling heads 24 and 26 have been placed, as in FIG. 2A, on opposite sides of the mesentery section 2, with the cooling faces opposite each other and aligned with each other.

Preferably, the probes are inserted endoscopically, through a portal or cannula inserted into the abdomen of the patient. With appropriate portal(s) in place, and appropriate insufflation applied to distend the abdomen and create working space (if necessary), the distal ends of probes which carry the cooling heads may be inserted through the portals and applied to the mesentery. Graspers with grasping jaws, or other retracting tools, can be inserted through the abdominal wall to hold the intestine and/or mesentery in a convenient configuration to facilitate application of the cooling heads. After the cooling operation, the cooling heads may be disengaged from the surfaces of the mesentery sheet. If the cooling operation has resulted in adhesion of the mesentery to the cooling heads, disengagement may be facilitated with active warming by applying energy to the heating elements, supplying warm fluid through the cooling fluid lumens, or waiting for passive warming to release the tissue from the cooling heads.

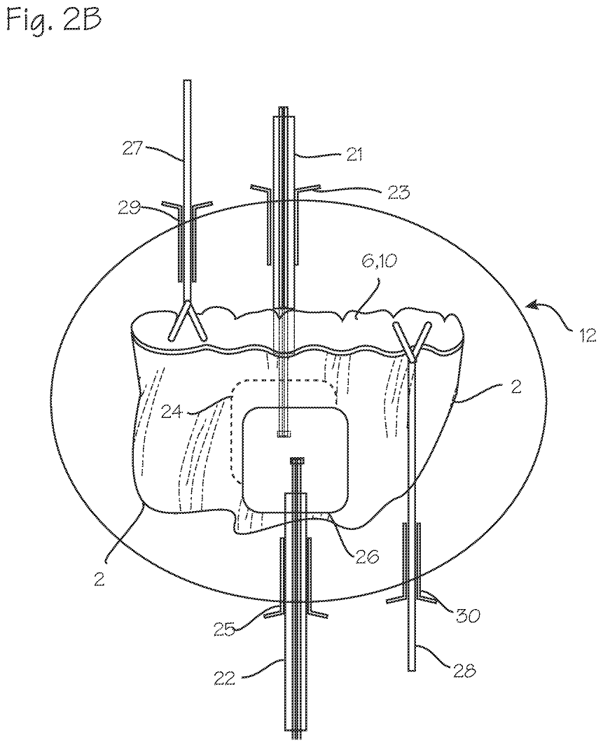

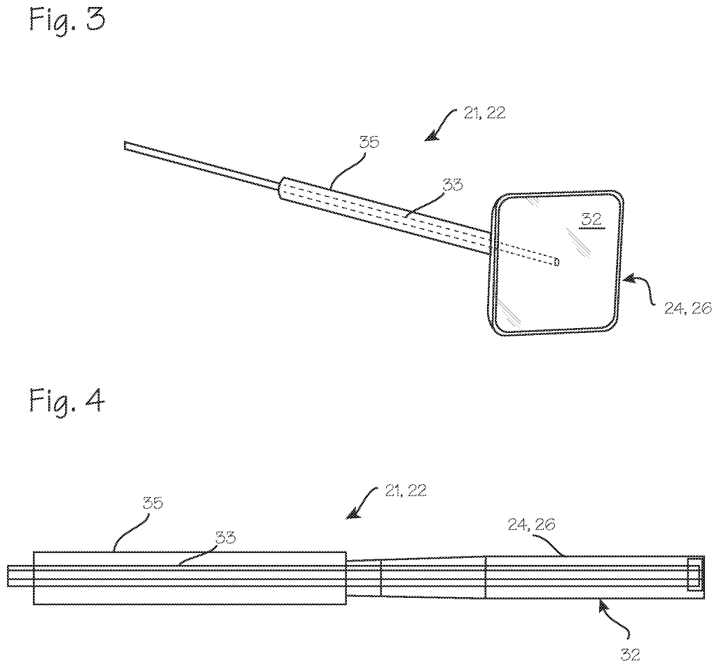

FIGS. 3 and 4 illustrates a cooling probe for use in the treatment method. FIG. 3 illustrates the flat distal surface 32 of the cooling probe (21 or 22) on the distal side of an expandable structure of the cooling head (24, 26), both disposed on the distal end of the cooling probe (21 or 22). FIG. 3 illustrates the cooling head in an expanded unconstrained configuration, which is achieved after insertion into the abdomen through the portals, while FIG. 4 illustrates the cooling head in a compressed constrained configuration, which is achieved for insertion through the portal. The flat distal face is suitable for treatment of the larger sheet-like mesenteric structures, such as the mesentery proper. For other mesenteric structures of differing geometry, the cooling heads may be provided in corresponding shapes, such as a concave surface on one cooling probe paired with a convex surface on the other cooling probe, or paired with a second concave surface. The pair of flat surfaces is merely the simplest case of a pair of distal surfaces configured to provide broad contact on opposing sides of a mesenteric structure.

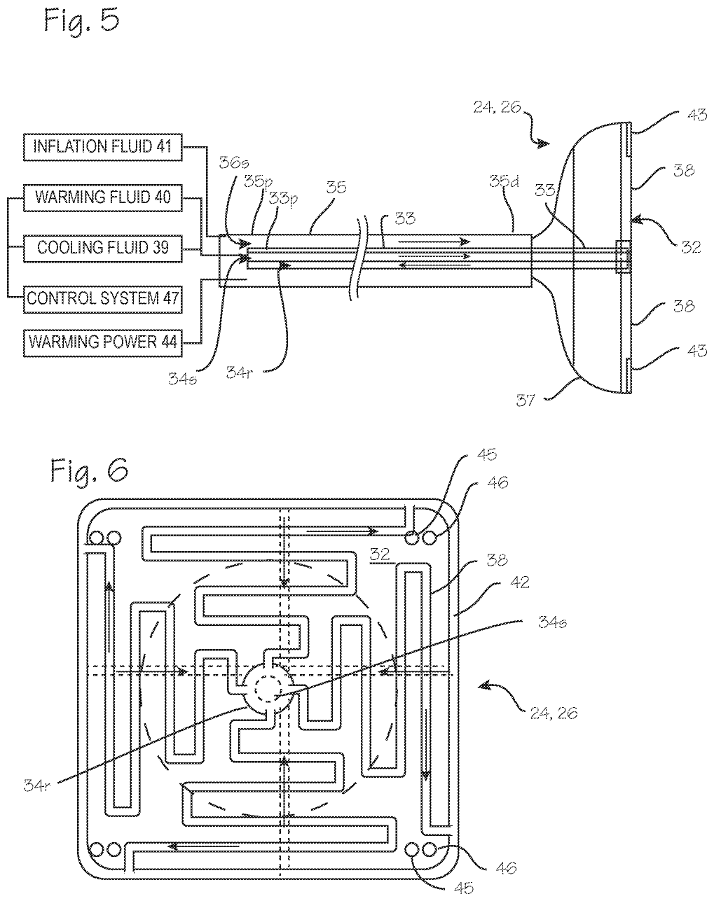

FIG. 5 is a cross section of the cooling probe. As shown in FIG. 5, the cooling probe (21 or 22) comprises an inner shaft 33 with a cooling fluid supply lumen 34s and a cooling fluid return lumen 34r extending from the proximal end 33p to the distal end 33d of the inner shaft and cooling head (24, 26) disposed at the distal end of the shaft. In embodiments in which the cooling head comprises an inflatable structure, the cooling probe also comprises an outer shaft 35 with an inflation fluid supply lumen 36s extending from the proximal end 35p to the distal end 35d of the outer shaft 35. The inflation fluid supply lumen is isolated from (not in fluid communication with) the cooling fluid lumens, such that the cooling heads may be inflated, and inflation fluid supplied, independently of the cooling fluid supply to the cooling channels in the cooling heads. The inflation fluid is preferably not provided at temperatures effective to treat visceral fat within the mesenteric structure. The cooling head preferably comprises an expandable and compressible structure 37, such as a balloon or resiliently expandable cage, with a distal surface which, in an expanded state of the structure 37 is substantially flat. The cooling head (24, 26) is, in the illustrated embodiment which is inflatable, sealed at its proximal end to the distal end 35d of the outer shaft. In mechanically expandable embodiments, the cooling head may be fixed to the distal end of the outer shaft without a fluid-tight seal. Cooling fluid channels 38 in communication with the supply and return lumens are provided on the distal surface of the cooling head, so as to apply cooling power to body tissue in contact with the distal surface. These cooling channels are in thermal communication with the cooling surface 32 of the probe, so that passage of cold fluid through the channels will cool tissue in contact with the cooling surface. At the proximal end of the cooling probe, the supply lumen 34s is configured for connection to a source of cooling fluid 39 (shown in FIG. 5). A warming fluid source 40 may also be provided, and configured for connection to the supply lumen 34s for use after cooling operations, to release the cooling head from frozen tissue. An inflation fluid source 41 in communication with the inflation lumen 36s is provided in those embodiments in which the expandable structure includes an inflatable structure. The cooling fluid, warming fluid, and inflation fluid reservoirs may be distinct, as depicted in FIG. 5, each in a physically distinct reservoir, or the same fluid may be used for two or all three of the fluids, and may be stored and drawn from the same reservoir (and cooled, warmed, or not, prior to delivery into the corresponding supply lumens).

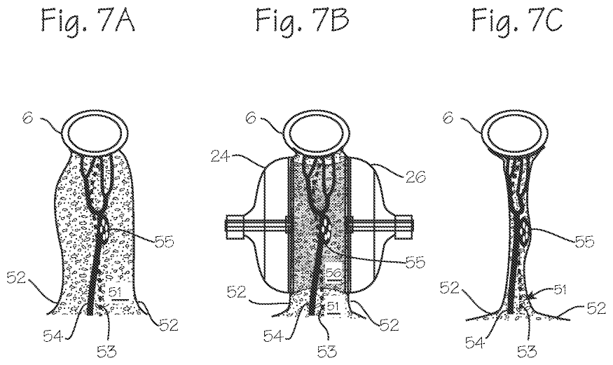

FIG. 6 illustrates the distal surface 32 of the cooling head (24, 26) of the cooling probe (21, 22). Various cooling fluid channels 38 are disposed proximate the surface, and are supplied with cooling fluid through the supply lumen 34. Cooling fluid flows through the channels to return manifold (or separate channels) 42 which is in fluid communication with the return lumen 34r of the shaft 33.

Warming elements 43 may be disposed on the distal surface, and wires for supplying electrical power to the warming elements may be disposed within the shaft and communicate with a power source outside the body. Warming elements, if provided, are connected to a power source 44 at the proximal end of the cooling probe.

Temperature sensors 45 may also be disposed on the cooling surface of one or both probes, and may be used by the surgeon to monitor the progress of the cooling operation and avoid under-cooling or over-cooling. Temperature sensors may also provide input to a control system, if used, which may be operable to control the flow and/or temperature of the cooling fluid to cool the tissue to the desired temperature. Otherwise, the surgeon may control the fluid flow manually, without the assistance of a control system which is operable to receive input from the temperature sensors and control fluid flow in response to the signals corresponding to temperature provided by the temperature sensors.

Position sensors 46 such as proximity sensors may be disposed on the cooling surface of each probe, and may be operable, in conjunction with a control system, to confirm that cooling heads on either side of a mesenteric structure are aligned with each other.

A control system 47 may be provided, programmed to control cooling fluid flow in response to operator input to initiate cooling, signals from the temperature sensors corresponding to the temperature of the visceral fat within the mesenteric structure, operator input to initiate warming, and also generate and output images to a display screen to provide information to the surgeon regarding the progress of the operations.

The return lumen 34r may be configured for connection to a collection tank for disposal or recirculation, or it may be open to atmosphere if the cooling fluid is a gas. The inner shaft may be rigid, so that it may be translated relative to the outer shaft to collapse the expandable structure when translated distally relative to the outer shaft and to expand, or facilitate expansion, of the expandable structure when translated proximally relative to the outer shaft.

The cooling fluid source 39 is operable to deliver cooling fluid through the cooling fluid supply lumen 34 and cooling fluid channels 38, to deliver cooling power to body tissue in contact with the distal surface. A warming fluid source 40, if provided, is operable to deliver warming fluid through the cooling fluid supply lumen 34 and cooling fluid channels 38, for use after the cooling operation, to deliver warming power to body tissue in contact with the distal surface. Alternately, the power source, if provided, is operable to deliver electrical power to the warming elements to apply warming heat to body tissue in contact with the distal surface, to release frozen tissue from the distal surface of the cooling head.

Any suitable cooling fluid may be used, including a solution of ethanol, ethanol in water, octafluoropropane, diethyl ether, or propylene glycol.

In use, a surgeon will access the abdomen by penetrating the skin and peritoneum 12 at one or more access ports, place appropriate portals in the penetrations, and insufflate the abdomen to create a working space, and insert an endoscope into the work space. The surgeon will insert appropriate retractors, such as the graspers 27 and 28 if necessary, retract abdominal organs as necessary to expose the mesentery sheet to the cooling probes, and insert cooling probes through portals into the abdomen. If the patient is in a prone position, the surgeon (or an assistant) will grasp the main organ to which the mesentery is attached (large intestine, small intestine, etc.) and lift it to allow the mesentery to hang down from the main organ, exposing the mesentery surfaces to the probes. (These steps are all optional, as the crux of the procedure may be accomplished with an open procedure, or with many variations in the steps of minimally invasive approaches.)

The surgeon will place one cooling probe on a first side of a mesentery structure (typically a sheet like structure) and place a second cooling probe on a second side of the mesentery structure, with the distal surfaces of the cooling heads of each cooling probe facing each other and aligned across the sheet, and press them together to ensure firm contact with the mesenteric structure. With the cooling heads aligned across the mesentery structure, the surgeon will initiate cooling fluid flow, from the cooling fluid source, through the supply lumen and channels in the distal faces of the cooling heads.

The surgeon will operate the cooling source and cooling probes to cool the mesenteric tissue trapped between the cooling heads to temperatures in the range of +10° C. to −60° C., and preferably in the range of +10° C. to −20° C., or the range of −20° C. to −40° C., and controlling the cooling probe to avoid cooling the visceral fat surrounding the distal end of the cooling probes to temperatures below about −40° C., to avoid cryogenic damage to blood vessels, nerves, lymph nodes and other structures within the mesentery, and the mesenteric membrane itself. In practice, it may be preferred to cool the tissue to a narrower range of +10° C. to −20° C. or −30° C., and avoiding cooling surrounding tissue below −30° C., and may be acceptable to cool the tissue to a broader range of +10° C. to −60° C., or more preferably to the range of −20° C. to −60° C., and avoiding cooling surrounding tissue below −60° C.

After cooling operations at one site, the cooling probes may be released from the mesenteric structure by warming the probe heads by energizing warming elements or passing warming fluid through the channels, or by passive warming with body heart. The process can be repeated to treat other areas of the mesentery. After the surgeon has treated one or more areas of the mesentery, the probes may be pulled from the abdomen, which may include collapsing the cooling heads by forcing a rigid inner shaft distally relative to the outer shaft, withdrawing or draining inflation fluid from the expandable structure, and pulling the probe through the portals.

The cooled visceral fat within the mesenteric structure is left in the body, to undergo cell death or elimination under any mechanism, within the mesenteric structure, to be resorbed by the body. The procedure may result in destruction and/or removal of the mesentery fat by processes including cryolipolysis (hydrolysis, cell disruption and inflammation), thermogenic fat metabolism without cell disruption, apoptosis (controlled cell death) or other natural process of the body.

For sufficiently thin mesenteric structures, both cooling heads may not be necessary to provide the cooling necessary for the treatment. If so, one of the cooling probes may be used in an inactive mode (operating a first cooling probe to cool tissue without operating the second probe to cool tissue disposed between the first distal surface of the first probe and the second distal surface of the second probe, for example), or may be replaced with a probe consisting of the expandable head, without cooling means, and this probe may be used as a backstop or anvil, for pressing the mesenteric structure into contact with the first cooling probe which will be operated as a cooling probe. Also, though the method is illustrated with probes with distal cooling surfaces suitable for trapping mesenteric tissue between two probes when the probes are inserted into the abdomen such the cooling heads can approach the mesenteric structure from opposite sides, the cooling surface can instead be disposed on a lateral surface of the cooling head, when, for example, the cooling heads might be disposed on opposing jaws of a clamp. Also, in cases where the surgeon cannot position device on either side of a mesentery structure, as in the case of the root of the mesentery, a single probe may be used, and operated independently of the second probe while pressing the cooling surface of the single probe against the root of the mesentery.

The results of the operation are illustrated in FIGS. 7A through 7C. FIG. 7A depicts a mesenteric sheet 2a attached to the intestine 6, with a thick layer of fat 51 between the mesenteric membranes 52, and nerves 53, blood vessels 54 and lymph nodes 55 within the fat. FIG. 7B shows the application of the cooling probe heads 24 and 26 and the extent of tissue 56 cooled by the probes. FIG. 7C shows the same portion of the mesenteric sheet after cooling and healing, in which much of the thermally treated fat has been absorbed and disposed of, or otherwise removed, by the healing process.

Thus, as described above, the method of removing visceral fat from a mesenteric structure within an abdomen of a patient, includes the steps of (1) trapping a portion of the mesenteric structure between a first cooling head of a first cooling probe and a second cooling head of a second cooling probe, (2) operating the first cooling probe to cool tissue within the mesenteric structure while operating the second cooling probe to cool tissue within the mesenteric structure, and (3) operating the second cooling probe to cool tissue within the mesenteric structure. The cooling heads are preferably operated at the same time, with cooling power being applied from both sides of the mesenteric structure simultaneously. The method is accomplished with first cooling head having a first surface configured for contacting the mesenteric structure (contacting the mesenteric membrane) and cooling the mesenteric structure, and a second cooling head having a second surface configured for contacting the mesenteric structure (contacting the mesenteric membrane) and cooling the mesenteric structure, and the step of trapping the portion of the mesenteric structure comprises placing the first surface in contact with the mesenteric structure and placing the second surface in contact with the mesenteric structure, with the distal surface of the first probe aligned across the mesenteric structure with the distal surface of the second probe. The portion of the mesenteric structure trapped between the cooling probes may be a single sheet, or a folded double layer of a mesenteric sheet, or a mass of different geometry. The cooling heads are cooled by forcing cooling fluid from a reservoir through the channels. When accomplished in an endoscopic/laparoscopic procedure, the method is accomplished by inserting the first cooling probe into the abdomen through a first portal, inserting the second cooling probe into the abdomen through a second portal, pressing the flat distal surface of the first cooling head on a first side of the mesenteric structure, pressing the flat distal surface of the second cooling head on a second side of the mesenteric structure, the second side of the mesenteric structure being opposite the first side of the mesenteric structure.

While the preferred embodiments of the devices and methods have been described in reference to the environment in which they were developed, they are merely illustrative of the principles of the inventions. The elements of the various embodiments may be incorporated into each of the other species to obtain the benefits of those elements in combination with such other species, and the various beneficial features may be employed in embodiments alone or in combination with each other. Other embodiments and configurations may be devised without departing from the spirit of the inventions and the scope of the appended claims.

We claim:

1. The method of removing visceral fat from a mesenteric structure within an abdomen of a patient, said method comprising the steps of:
trapping a portion of the mesenteric structure between a first cooling head of a first cooling probe and a second cooling head of a second cooling probe;
operating the first cooling probe to cool tissue within the mesenteric structure while operating the second cooling probe to cool tissue within the mesenteric structure.

2. The method of claim 1, wherein:
the first cooling head has a first surface configured for contacting the mesenteric structure and cooling the mesenteric structure, and the second cooling head has a second surface configured for contacting the mesenteric structure and cooling the mesenteric structure, and the step of trapping the portion of the mesenteric structure comprises placing the first surface in contact with the mesenteric structure and placing the second surface in contact with the mesenteric structure.

3. The method of claim 2 further comprising:
operating the first cooling probe and the second cooling probe to cool tissue disposed between the first surface of the first cooling head and the second surface of the second cooling head to temperatures effective treat visceral fat within the mesenteric structure yet ineffective to harm non-fat tissue within the mesenteric structure.

4. The method of claim 2, wherein:
the step of operating the first cooling probe to cool tissue comprises forcing cooling fluid through channels disposed in thermal communication with the first surface; and;
the step of operating the second cooling probe to cool tissue comprises forcing cooling fluid through channels disposed in thermal communication with the second surface.

5. The method of claim 1, further comprising:
inserting the first cooling probe into the abdomen through a first portal;
inserting the second cooling probe into the abdomen through a second portal;
disposing a first flat distal surface of the first cooling head on a first side of the mesenteric structure;
disposing the second flat distal surface of the second cooling head on a second side of the mesenteric structure, the second side of the mesenteric structure being opposite the first side of the mesenteric structure.

6. The method of claim 1, further comprising the steps of:
operating a warming means on the first cooling head, and operating a warming means on the second cooling head, to release the cooling heads from the mesenteric structure, after performing the steps of operating the cooling probed to cool tissue within the mesenteric structure.

7. The method of claim 1, wherein the step of operating the first cooling probe and the second cooling probe to cool tissue disposed between the first distal surface and the second distal surface comprises operating the first cooling probe and the second cooling probe to cool tissue disposed between the first distal surface and the second distal surface to temperatures in the range of +10° C. to −40° C. and controlling the cooling probe to avoid cooling the visceral fat surrounding the distal end of the cooling probes to temperatures below about −40° C.

8. The method of claim 1, wherein the step of operating the first cooling probe and the second cooling probe to cool tissue disposed between the first distal surface and the second distal surface comprises operating the first cooling probe and the second cooling probe to cool tissue disposed between the first distal surface and the second distal surface to temperatures in the range of +10° C. to −30° C. and controlling the cooling probe to avoid cooling the visceral fat surrounding the distal end of the cooling probes to temperatures below about −30° C.

9. The method of claim 1, wherein the step of cooling visceral fat comprises:
cooling the visceral fat to temperatures in the range of +10° C. to −60° C. while avoiding cooling the visceral fat to temperatures below about −60° C.

10. The method of claim 1, further comprising the step of:
leaving visceral fat, after cooling, in the body to be resorbed by the body.

11. The method of reducing visceral fat from a mesenteric structure within an abdomen of a patient, where said mesenteric structure has a first mesenteric membrane of the mesenteric structure on the first side of the mesenteric structure, and a second mesenteric membrane of the mesenteric structure on the second side of the mesenteric structure opposing the first mesenteric membrane,
said method comprising the steps of;
(1) applying a first distal surface of a first cooling probe to a first area of a first mesenteric membrane of the mesenteric structure on the first side of the mesenteric structure;
(2) applying a second distal surface of a second cooling probe to a second area of a mesenteric membrane of the mesenteric structure, second area being on the second side of the mesenteric structure; and
(3) operating the first cooling probe and the second cooling probe to cool tissue disposed between the first distal surface and the second distal surface to temperatures effective treat visceral fat within the mesenteric structure yet ineffective to harm non-fat tissue within the mesenteric structure, while holding the second distal surface proximate first distal surface on the second side of the mesenteric structure and pressing one of the first distal surface and the second distal surface toward the other one of the first distal surface and the second distal surface.

12. The method of claim 11, wherein the step of operating the first cooling probe and the second cooling probe to cool tissue disposed between the first distal surface and the second distal surface comprises:
operating the first cooling probe and the second cooling probe to cool tissue disposed between the first distal surface and the second distal surface to temperatures in the range of +10° C. to −40° C. and controlling the cooling probe to avoid cooling the visceral fat surrounding the distal end of the cooling probes to temperatures below about −40° C.

13. The method of claim 11, the step of operating the first cooling probe and the second cooling probe to cool tissue disposed between the first distal surface and the second distal surface comprises operating the first cooling probe and the second cooling probe to cool tissue disposed between the first distal surface and the second distal surface to temperatures in the range of +10° C. to −30° C. and controlling the cooling probe to avoid cooling the visceral fat surrounding the distal end of the cooling probes to temperatures below about −30° C.

14. The method of claim 11, wherein the step of cooling visceral fat comprises:
cooling the visceral fat to temperatures in the range of +10° C. to −60° C. while avoiding cooling the visceral fat to temperatures below about −60° C.

15. The method of claim 11, further comprising the step of:
leaving visceral fat, after cooling, in the body to be resorbed by the body.

16. The method of removing visceral fat a mesenteric structure within an abdomen of a patient, where said mesenteric structure has a first mesenteric membrane of the mesenteric structure on the first side of the mesenteric structure and a second mesenteric membrane of the mesenteric structure on the second side of the mesenteric structure opposing the first mesenteric membrane, said method comprising the steps of:
(1) applying a first distal surface of a first probe to a first area of a first mesenteric membrane of the mesenteric structure on the first side of the mesenteric structure, said first probe being a cooling probe operable, in conjunction with a cooling fluid source, to cool visceral fat within the mesenteric structure;
(2) applying a second distal surface of a second probe to a second area of a mesenteric membrane of the mesenteric structure, second area being on the second side of the mesenteric structure; and
(3) operating the first probe to cool tissue disposed between the first distal surface and the second distal surface to temperatures effective treat visceral fat within the mesenteric structure yet ineffective to harm non-fat tissue within the mesenteric structure, while holding the second distal surface proximate first distal surface on the second side of the mesenteric structure and pressing one of the first distal surface and the second distal surface toward the other one of the first distal surface and the second distal surface, without operating the second probe to cool tissue disposed between the first distal surface and the second distal surface.

17. A system for reducing visceral fat from a mesenteric structure within an abdomen of a patient, where said mesenteric structure has a first mesenteric membrane of the mesenteric structure on the first side of the mesenteric structure, a second mesenteric membrane of the mesenteric structure on the second side of the mesenteric structure opposing the first mesenteric membrane, said system comprising:
(1) a first cooling probe having a first cooling head with a first distal surface configured to contact a first area of a first mesenteric membrane of the mesenteric structure on the first side of the mesenteric structure;
(2) a second cooling probe having a second cooling head with a second distal surface configured to contact a second area of a second mesenteric membrane of the mesenteric structure on the second side of the mesenteric structure;
(3) a reservoir of cooling fluid; and
(4) a control system operable to force cooling fluid from the reservoir and into thermal communication with the first distal surface and the second distal surface, and control cooling fluid flow to cool mesenteric tissue disposed between the first distal surface and the second cooling surface to temperatures effective treat visceral fat within the mesenteric structure (destroy, ablate, induce cell death) yet ineffective to (destroy, ablate, induce cell death) non-fat tissue within the mesenteric structure.

18. A cooling probe for cooling mesenteric tissue, said cooling probe comprising:
an expandable and compressible cooling head disposed at the end of an elongate outer shaft, said outer shaft having an inflation lumen in fluid communication with the expandable cooling head, through which the cooling head may be inflated into a large, expanded configuration, said cooling head being compressible to a compressed configuration for insertion into a body through a portal;
said cooling head having a fluid channel proximate a distal surface of the cooling head, through which cooling fluid may be passed to cool the distal surface; and
an inner shaft, disposed within the outer shaft, said inner shaft having a cooling fluid supply lumen in fluid communication with the fluid channel, for supply of cooling fluid from a source of cooling fluid, from a proximal end of the inner shaft, through the lumen and through the cooling channels, said inner shaft having a return lumen in communication with the fluid channel, for return of the cooling fluid to the proximal end of the inner shaft; wherein
the inflation lumen is isolated from the cooling fluid supply lumen.

19. The cooling probe of claim 18, wherein the distal surface is substantially flat in an expanded, unconstrained configuration.

20. The cooling probe of claim 18, wherein the distal surface is convex or concave in an expanded, unconstrained configuration.
